(19) United States
(12) Patent Application Publication
(10) Pub. No.: US 2014/0375464 A1
(43) Pub. Date: Dec. 25, 2014

(54) Electronic Gas Sensor System and Methods of Operation

(71) Applicant: John Paul CARAGATA, Slocan, (CA)

(72) Inventor: John Paul Caragata, Slocan (CA)

(21) Appl. No.: 14/376,601

(22) PCT Filed: Feb. 8, 2013
(86) PCT No.: PCT/CA2013/000111

§ 371 Date: Aug. 4, 2014

Related U.S. Application Data
(60) Provisional application No. 61/597,018, filed on Feb. 9, 2012.

(51) Int. Cl. G01N 33/00

(52) U.S. Cl. CPC G01N 33/0063; G01N 33/0031; G01N 33/0075
USPC 340/632; 73/31.02

(57) ABSTRACT

The invention generally relates to systems and methods to monitor the presence of gases and particularly hazardous gases in and around equipment including drilling rigs. The system includes a gas detection system having at least one gas sensor for detecting hazardous gases around the equipment, individual conduits connected to each gas sensor for conveying gases from around the equipment to each gas sensor and a gas flow system to draw gases through the system. The system provides improved flexibility in the installation and adaptation of gas sensors to equipment such that hazardous gas detection and warning to personnel is improved.

### FIELD OF THE INVENTION

The invention generally relates to systems and methods to monitor the presence of gases and particularly hazardous gases in and around equipment including drilling rigs. The system includes a gas detection system having at least one gas sensor for continuously detecting the composition and concentration of gases around the equipment, individual conduits connected to each gas sensor for conveying gases from around the equipment to each gas sensor and a gas flow system to draw gases through the system. The system provides improved flexibility in the installation and adaptation of gas sensors to equipment such that hazardous gas detection and warning to personnel is improved.

### BACKGROUND OF THE INVENTION

In the oil and gas industry, during the drilling of a well, gases released from the formation being drilled are conveyed to surface where they can present significant safety problems to rig personnel and equipment.

Generally, as is well known, during drilling, gases and/or fluids are liberated from the formation rock which then becomes entrained in the drilling fluid that is being circulated through the well. As the drilling fluid is at a relatively higher pressure at the drill bit due to hydrostatic pressure forces from the overlying drilling fluid, as the drilling fluid returns to surface, the hydrostatic pressures are released and pressurized gases are liberated from the drilling fluid. Upon reaching the surface, as the drilling fluid is co-mingled with drill cuttings from the well, the drill cuttings must be separated from the drilling fluid to enable the drilling fluid to be re-circulated down the well. More specifically, the recovered solids and liquids will be subjected to various solids control methods and/or surface processes which also causes the release of additional and/or larger volumes of gases from the drilling fluid.

As the composition/concentration of released gases can be highly dangerous, at surface it is very important to quickly ascertain both the composition and concentration of the gases being released in order to ensure that all safety standards are met for both rig personnel and equipment.

Drilling rigs are large and complex assemblies of equipment and while there are typical forms and designs of drilling rigs, each job site is significantly different from one another in terms of the specific layout and/or use of particular equipment. As a result, the specific location and/or nature of a gas release will not necessarily be consistent across different drilling rigs. Further still, drilling rigs are also subjected to substantial changes in weather conditions as well as conditions controlled by mechanical equipment including radiator fans during their operation that can have a significant effect on the dissipation and/or detection of gases.

The monitoring of gas presence and gas concentration is well known at drilling rigs as it is legal requirement to ensure the safety standards imposed by different jurisdictions are being met. Generally, gas detection systems will be deployed at key locations at the drilling rig where it is known or expected that problems may occur. For example, solids control equipment is a key location where gas detection is required as this is usually the first location where drilling fluid is exposed to atmospheric pressures and thus is the location where the greatest volumes and concentrations of gas will be released. However, as is known, there are also other locations that may require gas monitoring.

Many different types of gas monitoring equipment can and have been deployed at drill sites and different manufacturers have created many different systems that may have been designed or developed to address particular requirements or functions including particular gas detection, sensitivity, responsiveness, deployment, alarms etc. While each particular manufacturer's piece of equipment may be effective for a particular task, the wide variety of equipment and systems presently being utilized and the differences between the various types of gas sensing equipment allows various inconsistencies to be manifested at a particular job site or between job sites. For example, operators installing different manufacturer's gas sensing equipment will often install the equipment with crude installation procedures in order to adapt a particular manufacturer's equipment to a particular job site. That is, often, an operator will crudely clamp, tape or otherwise attach the sensing equipment to rig equipment in locations where the operator simply deems appropriate. As a result, one operator at one rig may install equipment in an entirely location and/or manner than a different operator at a different rig. Such installations may or may not be within the manufacturer's guidelines for a particular piece of equipment. As can be appreciated, these inconsistencies may affect safety and may present additional problems to the operation of the rig.

Another problem is that different manufacturer's equipment will utilize different protocols for various functions including calibration, data display, programming, alarms etc. As a result, when different equipment is installed, it will require that personnel be specifically trained and/or understand the operation of each piece of equipment. As a result, the reliability and accuracy of data being obtained from these different pieces of equipment must be interpreted in the context of that particular piece of equipment. In other words, with many parameters being controlled according to different protocols, the fundamental baseline accuracy of data across all of the gas sensing equipment is substantially reduced.

Another problem is that, depending on the location where the gas sensing equipment is installed, it is well known that rig personnel may take various steps to crudely protect the sensors and/or alarm equipment by covering the equipment with various shields in order to protect the equipment from inadvertent impact and/or weather conditions. Such shields may include the use of scrap materials from around the work site to provide crude covers to the sensors. Clearly, the use of crude shields may significantly affect the reliability of a sensor.

A still further and significant problem is that rig personnel will also notice that familiar pieces of equipment are installed differently at different rigs which may often significantly affect their “comfort level” in the equipment that they are relying upon to keep them safe.

As can be appreciated, in view of the above reality in the field, there has been a need for a gas detection system that overcomes the above problems and that specifically improves the efficiency of installation as well as the effectiveness and accuracy of gas detection at a well site. Importantly, by achieving these objectives, the confidence of rig personnel in trusting the gas detection systems for monitoring their personnel safety is improved.

A review of the prior reveals that various gas sensing systems have been developed. Such patents include U.S. Pat. No. 4,138,891 (Graves et al.); U.S. Pat. No. 3,757,583 (Ludewig, Jr.); U.S. Pat. No. 6,470,760 (Shinozaki et al.); U.S. Pat. No. 6,241,950 (Veelenturf et al.); U.S. Pat. No. 7,389,704 (Desrochers et al.); U.S. Pat. No. 6,425,297 (Sharp); U.S. Pat. No. 3,357,257 (Herndon et al.); U.S. Patent Application Publication No. 2009/0244279 (Walsh); U.S. Pat. No. 4,565,086 (Orr, Jr.); U.S. Pat. No. 4,250,142 (Kollmai); and, U.S. Patent Application Publication No. 2011/0284288 (Sawyer et al.).

### SUMMARY OF THE INVENTION

In accordance with the invention, a system for monitoring gases at locations around equipment where hazardous gases may be present is described, the system comprising: at least one gas detection system having at least one gas sensor cell, each gas sensor cell having at least one gas sensor for detecting the presence of hazardous gases around the equipment; an individual conduit for operative connection to each gas sensor cell, each conduit for conveying gases directly from around the equipment to each gas sensor cell; a gas flow system operatively connected to each gas sensor cell and conduits for continuously moving gases through the conduits from the equipment to each gas sensor cell; and a controller operatively connected to each gas detection system for controlling the gas detection system and for receiving data signals from the gas sensors and gas flow system.

In further embodiments, the controller has means to simultaneously monitor gas flow rates through each gas sensor cell and the presence of the hazardous gas within each gas sensor cell and/or the controller includes means to adjust the flow rate of gases through each gas sensor cell based on monitored gas flow rates within each gas sensor cell and wherein the gas flow system and controller includes means for increasing or decreasing the flow rate of gases through each gas sensor cell.

In another embodiment, each gas sensor cell includes an independent gas flow system for moving gases through the conduits to each individual gas sensor.

In further embodiments, the system further comprises an alarm system operatively connected to the controller and wherein the controller includes means for providing an audio and/or a visual alarm if the controller detects an alarm condition. The alarm system may provide distinct visual alarms that correspond to each gas sensor cell.

In another embodiment, the alarm system includes at least one remote alarm module having a wireless communication means for communicating with the controller.

In one embodiment, the at least one remote alarm module includes a body having a plurality of visual indicators distributed about the remote alarm module enabling viewing of the visual indicators through at least a 270 degree field of view.

In a further embodiment, each alarm module includes an LED light array having means for displaying the severity and location of an alarm signal from at least two sensor cells.

In another embodiment the gas detection system is modular and includes a base enabling individual gas sensor cells to be individually connected to the base, thereby allowing a customized gas detection system to be assembled.

In yet another embodiment, the gas detection system further comprises a standalone gas sensor module for configuration to equipment, the standalone gas sensor module having at least one gas sensor, gas conduit and gas flow system and wherein the individual gas sensor module communicates gas sensor data to the controller by a wired or wireless link.

In another embodiment, the controller has means to provide an alarm signal in the event that the flow of gas through a conduit drops below a threshold level.

In another embodiment, the system includes a display system operatively connected to the controller for displaying real time data from the gas detection system. The display system may be operatively connected to the controller for displaying real time data from the gas flow system. The alarm system may also at least one speaker and the controller may also include means for generating voice commands through the at least one speaker.

To facilitate attachment of components of the system to equipment, conduits and remote alarm modules may include magnets. The remote alarm module may also include at least one camera mount for attaching a remote camera to the remote alarm module such that the remote alarm module and controller can respectively send and receive image data between the remote alarm module and the controller.

In one embodiment, the gas flow system includes an exhaust system for exhausting gases from the system and the exhaust system includes at least one conduit for conveying gases away the system to a designated location. The exhaust system may also include at least one gas sensor operatively connected to the controller for monitoring hazardous gases within the exhaust system.

In another aspect, the invention provides a system for monitoring gases at locations around equipment where hazardous gases may be present, the system comprising: at least one modular gas detection unit having: at least one gas sensor cell having at least one gas sensor for detecting the composition and concentration of gases around the equipment; a conduit operatively connected to each modular gas sensor cell for conveying gases from around the equipment to the gas sensor cell; a gas flow system operatively connected to each gas sensor cell for moving gases through the conduit to each gas sensor cell; and, a controller operatively connected to the modular gas detection unit for controlling the modular gas detection system and for receiving data signals from each gas sensor. The at least one modular gas detection unit and controller may each have wireless communication means for communicating data between the at least one modular gas detection unit and controller.

In another aspect, the invention provides a pre-assembled plenum for configuration to equipment to detect hazardous gases around the equipment, the pre-assembled plenum comprising at least two individual gas sensor cells operatively mounted within a plenum body, the gas sensor cells including: at least one gas sensor for measuring hazardous gas within a sensor cell; an inflow conduit connected to each sensor cell for drawing gases into a sensor cell, the inflow conduit extending from the plenum; an outflow conduit connected to each sensor cell for expelling gases from a sensor cell, the outflow conduit extending from the plenum; wherein each of the inflow and outflow conduits include means for connecting conduits to the inflow and outflow conduits. In one embodiment, an individual gas flow system is operatively connected to each sensor cell for drawing gas through each sensor cell.

In a still further aspect, the invention provides a method of detecting hazardous gases around equipment comprising the steps of: a) conveying gases from around the equipment through a conduit to a gas detection system having at least one modular gas sensor cell having at least one gas sensor for detecting the composition of gases around the equipment wherein gases are conveyed through separate conduits to each modular gas sensor cell through separate conduits; b) determining the composition of the gases from around the equipment and c) initiating an alarm if an alarm condition is detected.

The method may also include the step of displaying or producing the alarm signal at the location where the hazardous gas is conveyed from. In one embodiment, gases are conveyed from at least two locations around the equipment and the step of displaying or producing the alarm signal at the location where the hazardous gas is conveyed from includes displaying a corresponding location of the origin of the alarm signal.

### BRIEF DESCRIPTION OF THE DRAWINGS

The invention is described with reference to the accompanying figures in which:

FIG. 1 is a schematic overview of a drilling rig incorporating the gas detection system in accordance with one embodiment of the invention;

FIG. 2 is a schematic overview of a modular gas detection system in accordance with one embodiment of the invention;

FIG. 2A is a schematic overview of a modular gas detection system in accordance with a second embodiment of the invention;

FIG. 3 is a schematic overview of a modular gas detection system in accordance with one embodiment of the invention showing individual sensor modules;

FIG. 4 is a schematic overview of the modular gas detection system configured to a typical drilling rig in accordance with one embodiment of the invention;

FIG. 5 is an alternate embodiment of a modular gas detection system configured to a typical drilling rig in accordance with one embodiment of the invention;

FIG. 6 is a schematic diagram of an alarm unit in accordance with one embodiment of the invention;

FIG. 6A is a schematic diagram of an alarm/display unit in accordance with a second embodiment of the invention;

FIG. 7 is a schematic diagram of a sensor unit having a self-contained gas flow system and a wireless communication interface;

FIG. 8 is a schematic diagram of a conduit in accordance with one embodiment of the invention; and,

FIG. 8A is a schematic diagram of an intake and trap in accordance with one embodiment of the invention.

### DETAILED DESCRIPTION OF THE INVENTION

With reference to the figures a gas detection system for continuously detecting the presence of potentially hazardous gases or concentrations of hazardous gases (collectively “hazardous gases”) in and around industrial equipment is described. More specifically, the system provides means for effectively conveying hazardous gases to a central detection module having a plurality of sensor units. The system is designed with modular components to provide a greater degree of flexibility in integrating the system across a variety of installations while improving the accuracy and responsiveness in the detection of hazardous gases.

The invention is described in the context of a drilling rig and associated equipment although it is understood that the system can be deployed with other equipment as understood by those skilled in the art.

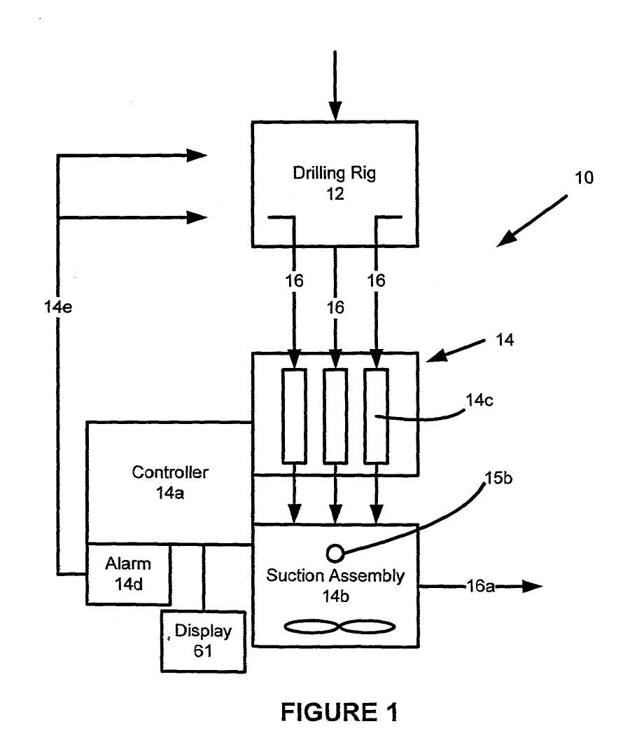

As shown in FIG. 1, a schematic diagram of the gas detection system (GDS) 10 as it may be configured to a drilling rig 12 is described. The GDS generally includes a gas detection unit (GDU) 14 having an associated controller 14a, display 60, suction assembly 14b and conduits 16. In accordance with the invention, hazardous gases originating around or within the drilling rig and its associated equipment are continuously conveyed through conduits 16 to the GDU 14 wherein individual sensor units 14c detect the composition and concentration of gases within each conduit. Gas composition and concentration information is interpreted within controller 14a and wherein when necessary, an alarm 14d is triggered which may be delivered as an alarm signal 14e to the location where the alarm was triggered and/or to any number of locations at the rig. Gases are drawn through the GDU by a suction assembly 14b. Waste gas is conveyed from the system through waste conduit 16a.

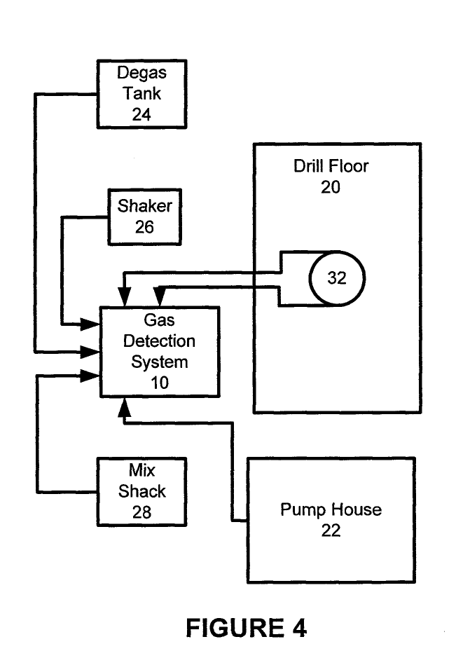

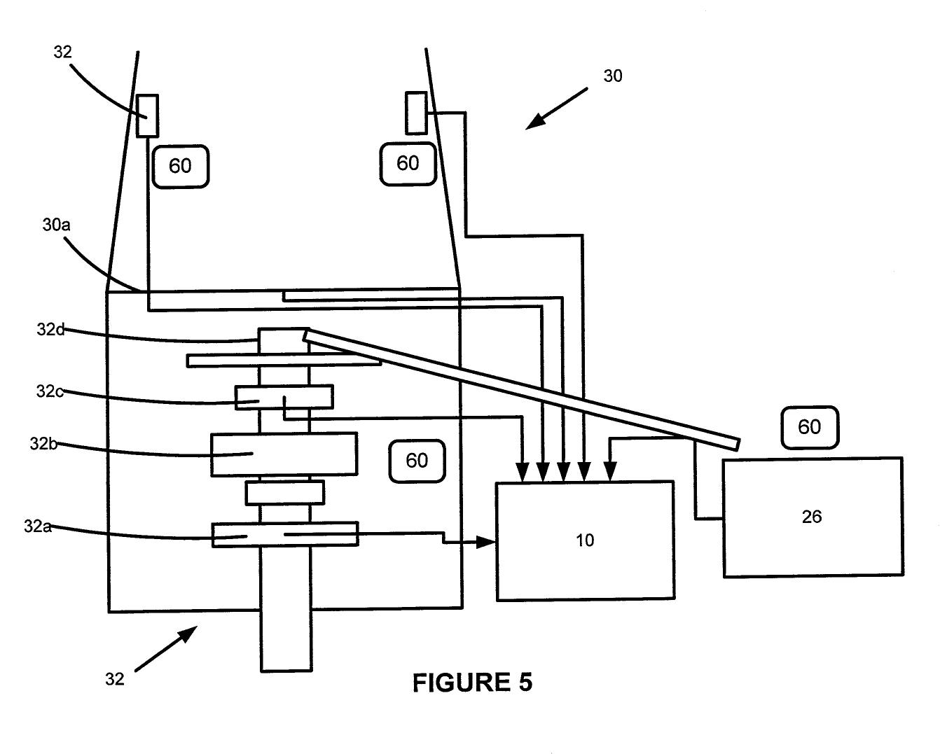

The GDS 10 can be configured to any number of locations at the well site where there is a potential for hazardous gases to be released. As shown in FIGS. 4 and 5, these locations may include the drill floor 20, pump house 22, degas tanks 24, shaker 26 and/or mix shack 28. As shown in FIG. 5, additional gas sampling locations may more specifically include locations around the drilling rig 30 and well head 32. These locations may include at or around the rig floor 30a, well head sub-structure 32a, blow-out preventer (BOP) stack 32b, bag/hydril 32c, flow nipple 32d and shaker 26. As understood by those skilled in the art, based on the specific layout/configuration of the drill site equipment that additional locations at the drilling site be configured with conduits 16 to effect transfer of gases to the GDU 14.

### Gas Detection Unit

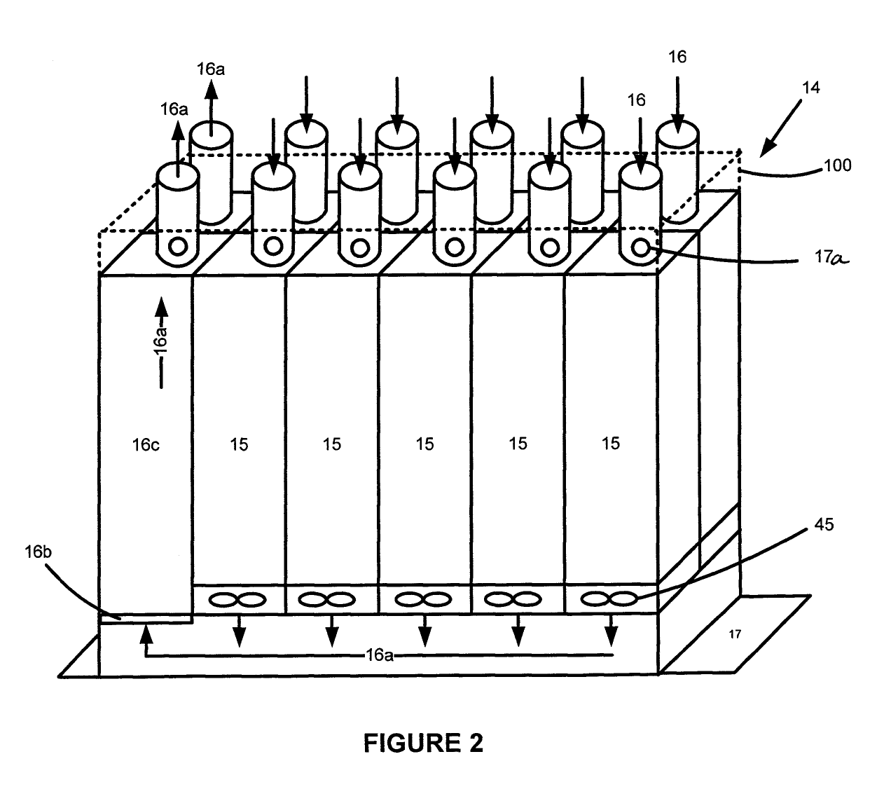

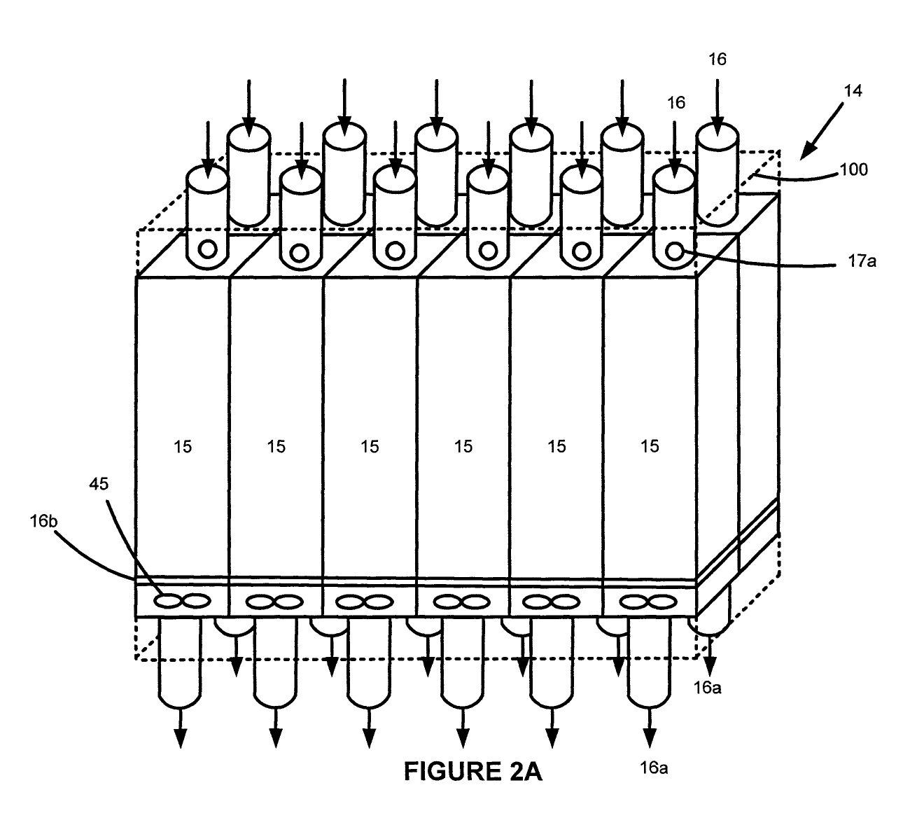

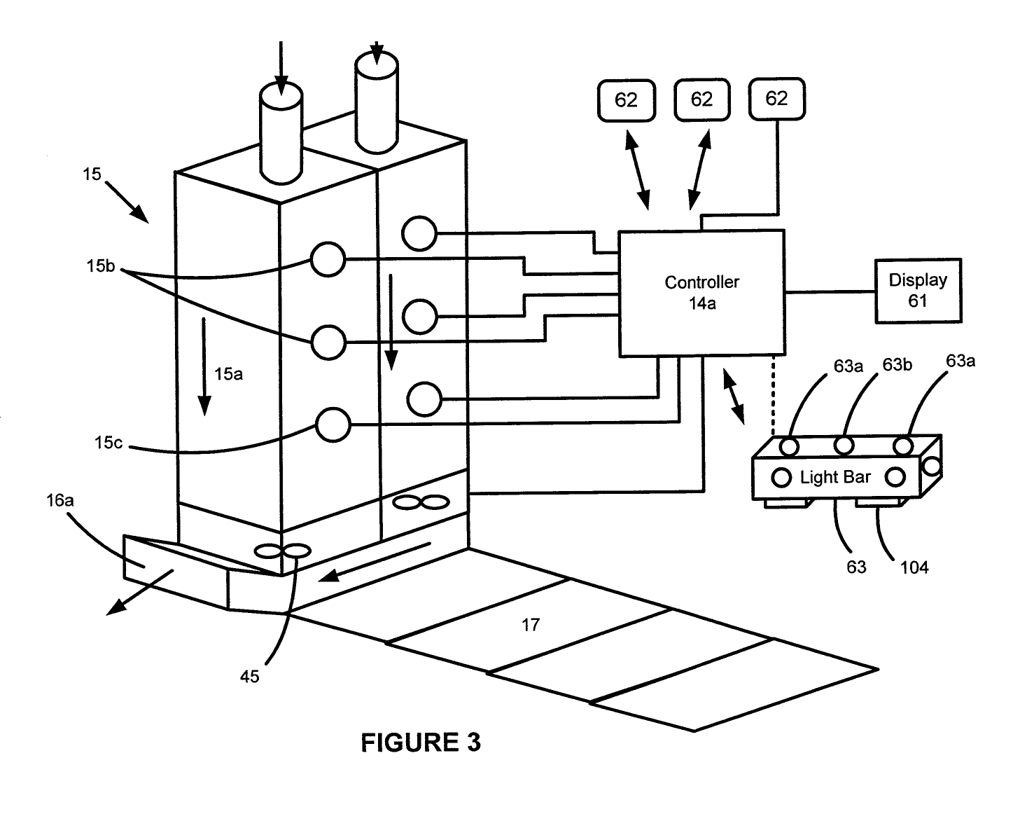

The GDU 14 is best shown in FIGS. 2, 2A and 3. A GDU generally includes at least one sensor cell 15 and preferably a plurality of sensor cells, each capable of individually monitoring and detecting the presence and concentration of gases received within each cell from a corresponding conduit 16. Generally, as shown in FIG. 3, each sensor cell 15 includes a sensor chamber 15a with one or more sensors 15b in operative communication with gases within the sensor chamber 15a. As such, as gases are conveyed into the sensor chamber 15a, the sensors 15b monitor and detect the presence of and/or concentration of a particular gas and provide an appropriate signal to controller 14a. Each sensor cell 15 is configured to either an individual or common waste conduit 16a that conveys gases away from each sensor chamber 15a. One or more gas sensors 15b may also be provided within the suction assembly 14b as shown in FIG. 1 in order to monitor the hazard status of the individual and/or collective exhaust gas.

In one embodiment, each sensor cell also includes an individual and separately controllable fan 45 that draws air through the sensor cell as well as a gas flow sensor 15c. The gas flow sensor provides information to the controller to verify that there is adequate air flow through the sensor cell. That is, in the event that dust, debris or moisture is preventing sufficient air flow through the sensor cell, the controller can interpret and advise that inaccurate gas readings may be being received from a particular location at the drill rig. Based on the gas flow readings being received, the controller may increase or decrease the speed of fan 45 to maintain the gas flow rates within preferred ranges.

As shown in FIG. 2, in one embodiment, the sensor chambers 15a together with waste conduit 16a may be configured to a base 17 allowing individual, pairs or other sensor assemblies to be interconnected in a combined package. In this case, a total of 10 individual sensor cells 15 are interconnected on a base 17 with a common waste conduit 16a conveying gases away from each sensor cell 15. In this example, the common waste conduit 16a forms the base that allows individual sensor cells to be attached to the base. In addition, the GDU includes an end module 16c that serves as a component of the GDU. As shown, the waste conduit 16 may include a filter 16b to enable filtering of particulates and/or moisture from the system. Filters 16b may also be configured to individual sensor cells as shown in FIG. 2A. Preferably, the assembled sensor cells are housed in an explosion proof cabinet 100 (shown schematically in dotted lines) as may be required within a jurisdiction. Also as understood to those skilled in the art, the cabinet 100 also includes appropriate electrics and connectors to provide power to the system, provide command inputs to the system from the controller and to receive data from the system.

A gas sample port 17a may be provided to enable operators to periodically test the system by injecting a sample of gas into each sensor cell as part of a sensor function test.

As shown in FIG. 2A, in an alternate embodiment, a pre-assembled plenum containing a plurality of sensor cells may be configured with a cabinet 100 to facilitate installation at the rig. In this embodiment, a number of sensor cells would be combined in a pre-assembled package wherein a number of inlet 16 and exhaust 16a conduits would be extend into and out of the cabinet. As shown, in this case, the cabinet 100 may be configured to a wall with inlet conduits 16 flowing into the top of the cabinet and waste conduits 16b extending from the bottom of the cabinet. In this embodiment, as shown, each sensor cell 15 includes an independently controllable fan 45.

The design and interconnection of individual sensor cells 15, waste conduits 16a, base 17 and other components can be varied as understood by those skilled in the art and may be adapted to specific operational requirements in different industries. For example, each of conventional oil, gas and/or fracturing operations may utilize different sensor cells for detecting different gases as may be required for the specifics of each industry.

As noted above, the GDU will include appropriate connectors to enable individual sensor cells 18 to be connected to inflowing conduits and to the exhaust system. As well, each sensor cell will have appropriate connectors to connect the sensors 15b to the controller 14a. As such, in one embodiment, as shown in FIGS. 2 and 3, a plurality of sensor cells can be assembled into a multi-channel sensor array giving the operator the flexibility to build a system having the desired number of channels deriving gas data from a plurality of rig locations. In addition, as a result of the modular nature of the sensor cells, in the event of failure of an individual cell, a replacement cell can be readily inserted into the array.

Preferably, each sensor cell 15 will include a duplicate sensor to increase the reliability of the gas measurements within the sensor chamber 15a.

Sensors may be selected from known gas sensors. In the rig example, sensors for detecting hydrogen sulfide and/or lower explosion limits (LEL) of gases may be typically deployed sensors.

### Conduits

The conduits 16 may be any suitable conduit effective in conveying gases from a rig location to the GDU 12. The conduits will typically be polymeric piping systems such as polyvinylchloride or polyethylene tubing that has sufficient rigidity to minimize the risk of conduit collapse. In one embodiment, the conduits are sufficiently flexible to allow the conduits to be coiled for deployment from a reel system during installation/deployment of the system. Industrial vacuum hose may also be used. In one embodiment, as shown in FIG. 8, the conduits 16 may include rigid/semi-rigid of sections of piping that include a plurality of magnets 104 enabling the placement of sections of conduits to the rig equipment. The magnets may be moveable with respect to a section of conduit to facilitate their installation.

In addition, each conduit will have appropriate connectors to allow end connectors 32 to be configured to the end each conduit. Each end connector 32 (FIGS. 5, 8A) may be specific to the desired location of deployment. For example, end connectors that are configured to rig floor locations may be provided with weather shields to minimize the risk of rain/snow being drawn into the conduits, whereas other end connectors that may be deployed within equipment may have appropriate filter/traps to reduce the risk of dust/moisture entering the conduits. As shown in FIG. 8A, the end connectors 32 may also be provided with filters and/or traps 102 adjacent the end of the connectors to enable removal of dust or moisture that may enter the conduits. Traps/filters may also be positioned at other locations in the conduits.

As shown in FIG. 8A, end connectors 32 may be also be provided with a double “U” design to ensure that moisture/debris is less likely to enter a conduit through an inverted “U” entrance while also providing a trap 102 to collect moisture/debris that may enter the system. The end connectors 32 may also include magnets 104 that may also assist in the installing the system.

### Controller

The controller 14a primarily interprets data received from the sensors 15b, 15c and provides appropriate signals to a display and/or alarm system. For example, a continuous digital output indicating the concentration of particular gas within a particular sensor cell is displayed at the controller. If a threshold value is reached, the controller may activate an alarm. If an alarm is triggered, in addition to providing a visual and/or audio signal, the controller may also initiate secondary actions including the automatic shutdown of equipment and/or the activation of secondary safety equipment. The controller may be connected to other local or remote display/control equipment using wired and/or wireless networks across a local or wide area network.

Importantly, the controller may also control the rate of flow of gas through each cell as may be required and increase or decrease the flow rate of gas through each conduit based on flow rate information.

The controller may also monitor the composition of exhaust gases in either individual or common exhaust conduits to ensure that the exhaust gases are not in themselves presenting a hazard. Exhaust conduits may be configured to ensure that all exhaust gases are conveyed to designated locations at a job site to ensure proper handling of the exhaust gases.

The controller may also detect and display fault data originating from the sensor cells as well as controlling calibration of the system.

In another embodiment, as shown in FIGS. 3, 5 and 6, the system may include one or more alarm display modules 62 operatively connected to the controller that display the alarm signals to the specific detection sites. Each alarm display module will preferably include a visual and audio alarm system that will provide different alarm levels 60a, 60b, 60c depending on the nature of the alarm event. The alarm display may also include an appropriate wireless interface 60d and battery system 60e if a standalone, wireless unit is desired.

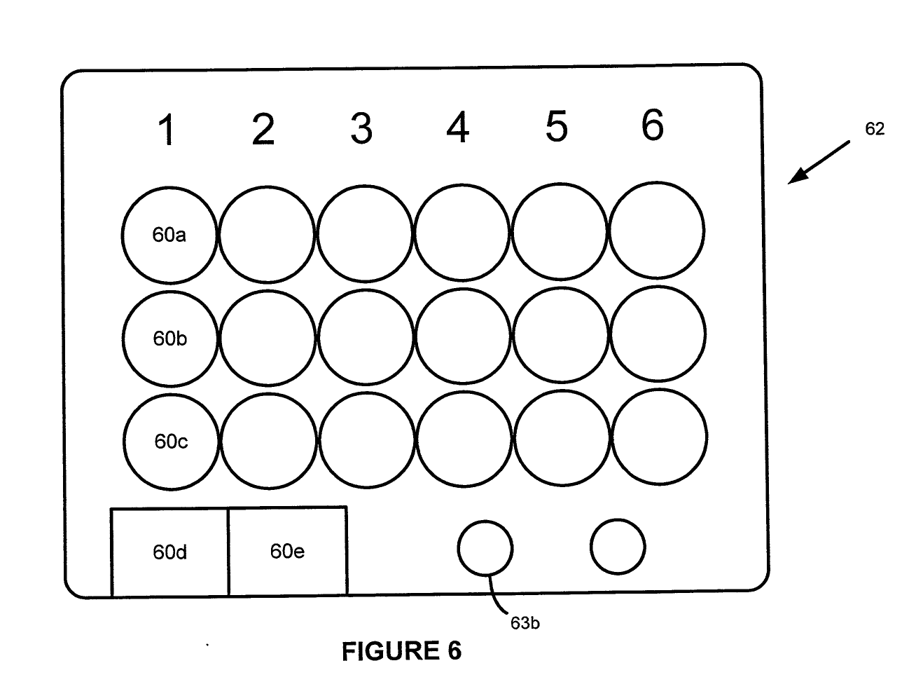

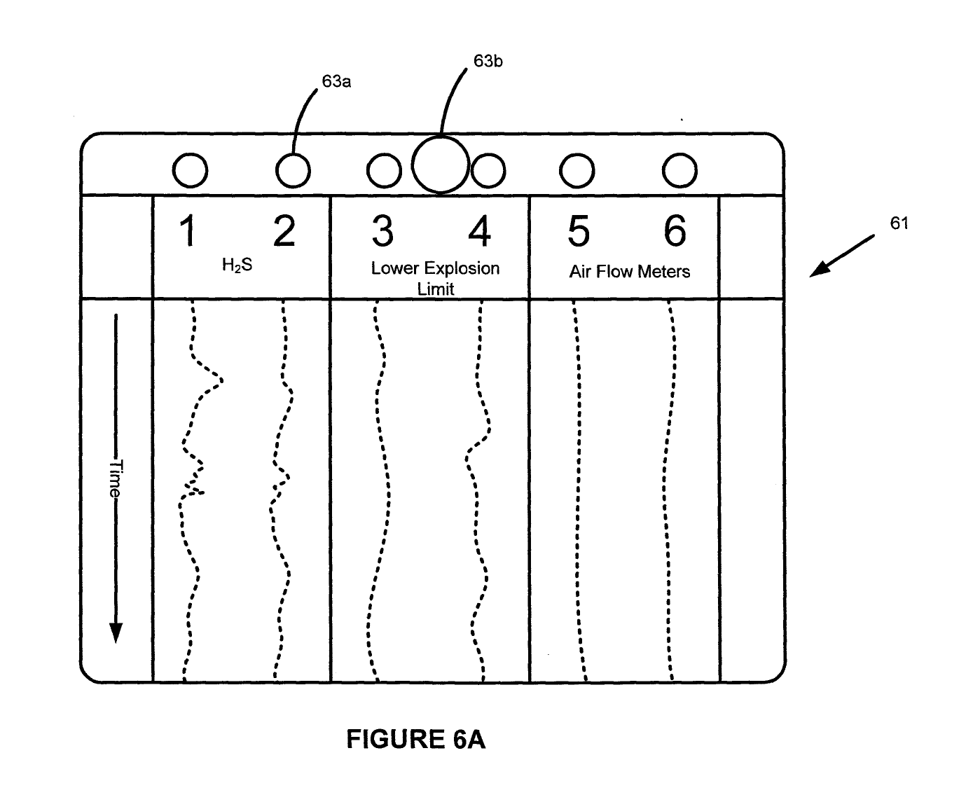

As shown in FIG. 6, in a preferred embodiment, each alarm display module will preferably include a display that displays alarm signals from a plurality of controller channels. For example, an array of LED lights (with corresponding audio alarms) may signal the location and severity of an alarm signal at each of six channel locations. In this case, a low level alarm at location 1 may trigger alarm light 60a. As the severity of the trigger signal increases, additional lights 60b and 60c may be turned on with corresponding audio alarms. Importantly, in this embodiment, rig personnel can be alerted to alarm signals not only at their specific location but they are also made aware of alarms originating from other locations. As understood, each channel on the alarm display module may be labeled with a simple channel number or provided with a specific location label.

In other embodiments, different alarm sequences may be initiated. In one representative alarm sequence, the alarm display module displays various light colors that provide additional information to personnel. For example, an alarm display module may display different colors representing each channel where a hazardous gas detected at different locations at rig would activate a different color. Additional colors within each channel may be activated to signal that a hazardous gas has been detected at each of the sensors within the sensor cells. Further colors may also be used to signal the relative concentration of the hazardous gas. In another embodiment, the controller may send a voice command signal to each alarm display that may identify the location of a hazard, the type of hazard and instructions. For example, a voice command may be broadcast through each alarm display module stating “Explosion hazard on rig floor; evacuate area”. Such voice commands may be programmed into the controller but may also be broadcast from the controller by an operator who is monitoring the equipment. In this case, the controller may include appropriate voice input and processing hardware/software.

In an alarm scenario, when a gas hazard has moved into one of the conduits, the lights on the alarm module become active. For example, a purple light may initially start to flash signifying that a hazardous gas has been detected on the rig floor, then a yellow light indicating that a hazardous gas has been detected at one of the sensors and a red light indicating that a hazardous gas has been detected at second of the sensors. The activation of the two sensors confirms the alarm and initiates an audible alarm and a corresponding flashing light for the area/channel where the alarm has occurred.

Importantly, using two sensors to verify an alarm reduces the number of false alarms as an alarm is only initiated when it has been verified. This improves the trustworthiness of the system and gives the personnel greater confidence in the system.

As shown in FIG. 3, in one embodiment an alarm unit 63 may be wired or wirelessly connected to the controller 14a. In this embodiment, the alarm unit may be designed as a light bar having a plurality of lights 63a on each side of the body of the unit. In this form, alarm signals will be visible from a number of directions and preferably with a field of view greater than 270 degrees. In addition, the alarm unit 63 may include one or more speakers 63b enabling voice messages to be broadcast as described above. In a preferred embodiment, the light bar includes one or more magnets 104 allowing the alarm unit be conveniently secured to rig equipment.

### Other Embodiments

The system may be designed to include a test gas bottle for use in calibration and testing of each sensor cell during set-up. In this embodiment, a sample of the hazardous gas may be deployed into the sensor cell through port 17a to verify the operability of the sensor and alarm scenarios.

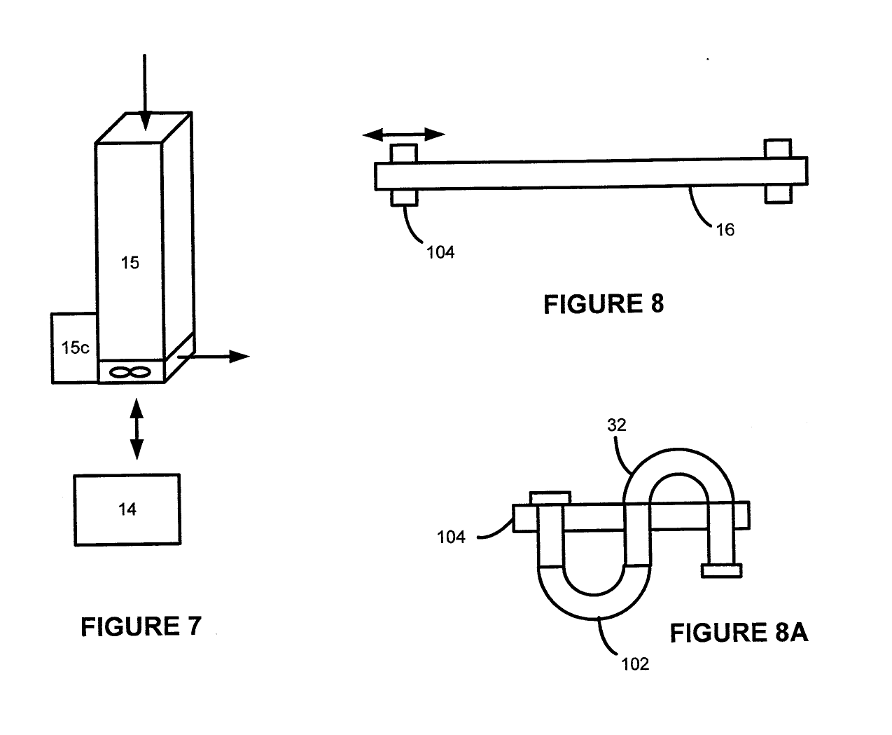

In another embodiment, as shown in FIG. 7, sensor cells may be standalone units having a wired or wireless interface enabling sensor data to be transmitted to a controller 14 and wherein the conduit 16 does not feed to a centralized sensor module. In the typical deployment scenario, conduits in the range of 5-10 m in length will be required and in this case, it will be generally be preferred that conduits are continuous. In deployments where longer lengths of conduit may be required, the wireless system may be advantageous so as to eliminate the need for running long lengths of conduit. In this case, a sensor cell may be provided with an integral power and control unit 15c and fan that enable wired or wireless transmission of data from the sensor unit to the controller 14 and drawing gas through the sensor cell.

Each of the alarm units 62, 63 may also be provided with cameras so as to enable an operator to monitor a gas sensing location.

### Operation and Deployment

The system can be readily installed at a drilling rig by assembly of the desired number of sensor cells (channels) to a controller and the deployment and connection of conduit from the sensor cells to the various locations at the rig. Typically, the conduit 16 will be unwound from a source reel such that one end is at the desired position on the drilling rig or be assembled from a number of conduit sections. The conduit may be fixed into place using an appropriate anchor system such as plastic ties, cables or wires as well as magnets as described above. End connectors may be connected to the ends of each conduit using any suitable connection system. Ideally, conduits and connectors will also allow disassembly and re-use of those connectors.

Color coding of conduits may also be implemented as well as distinct alarms associated with each conduit color such that rig personnel can learn to associate specific alarms with specific locations on the rig.

Importantly, the system, by virtue of the modular components permits the operators the flexibility to design and deploy the system quickly and with a high degree of consistency between different sites.

Although the present invention has been described and illustrated with respect to preferred embodiments and preferred uses thereof, it is not to be so limited since modifications and changes can be made therein which are within the full, intended scope of the invention as understood by those skilled in the art.

1. A system for monitoring gases at locations at atmospheric pressure around equipment where hazardous gases may be present, the system comprising:
at least one gas detection system having at least one gas sensor cell, each gas sensor cell having at least one gas sensor for detecting the presence of hazardous gases around the equipment;
an individual conduit for operative connection to each gas sensor cell, each conduit for conveying gases directly from around the equipment to each gas sensor cell;
a gas flow system operatively connected to each gas sensor cell and conduits for continuously moving gases through the conduits from the equipment to each gas sensor cell;
a controller operatively connected to each gas detection system for controlling the gas detection system and for receiving data signals from the gas sensors and gas flow system.

2. The system as in claim 1 wherein the controller has means to simultaneously monitor gas flow rates through each gas sensor cell and the presence of the hazardous gas within each gas sensor cell.

3. The system as in claim 2 wherein the controller includes means to adjust the flow rate of gases through each gas sensor cell based on monitored gas flow rates within each gas sensor cell and wherein the gas flow system includes means for increasing or decreasing the flow rate of gases through each gas sensor cell.

4. The system as in claim 1 wherein each gas sensor cell includes an independent gas flow system for moving gases through the conduits to each individual gas sensor.

5. The system as in claim 1 further comprising an alarm system operatively connected to the controller and wherein the controller includes means for providing an audio and/or a visual alarm if the controller detects an alarm condition.

6. The system as in claim 5 wherein the alarm system provides distinct visual alarms that correspond to each gas sensor cell.

7. The system as in claim 5 wherein the alarm system includes at least one remote alarm module having a wireless communication means for communicating with the controller.

8. The system as in claim 7 wherein the at least one remote alarm module includes a body having a plurality of visual indicators distributed about the remote alarm module enabling viewing of the visual indicators through at least a 270 degree field of view.

9. The system as in claim 5 wherein each alarm module includes an LED light array having means for displaying the severity and location of an alarm signal from at least two sensor cells.

10. The system as in claim 1 wherein the gas detection system is modular and includes a base enabling individual gas sensor cells to be individually connected to the base.

11. The system as in claim 1 wherein the gas detection system further comprises a standalone gas sensor module for configuration to equipment, the standalone gas sensor module having at least one gas sensor, gas conduit and gas flow system and wherein the individual gas sensor module communicates gas sensor data to the controller by a wired or wireless link.

12. The system as in claim 2 wherein the controller has means to provide an alarm signal in the event that the flow of gas through a conduit drops below a threshold level.

13. The system as in claim 1 further comprising a display system operatively connected to the controller for displaying real time data from the gas detection system.

14. The system as in claim 13 further comprising a display system operatively connected to the controller for displaying real time data from the gas flow system.

15. The system as in claim 5 wherein the alarm system includes at least one speaker and the controller includes means for generating voice commands through the at least one speaker.

16. The system as in claim 7 wherein the remote alarm module includes at least one magnet for attaching the remote alarm module to equipment.

17. The system as in claim 7 wherein the remote alarm module includes at least one camera mount for attaching a remote camera to the remote alarm module and wherein the remote alarm module and controller can respectively send and receive image data between the remote alarm module and the controller.

18. The system as in claim 1 wherein the gas flow system includes an exhaust system for exhausting gases from the system and the exhaust system includes at least one conduit for conveying gases away the system to a designated location.

19. The system as in claim 18 wherein the exhaust system includes at least one gas sensor operatively connected to the controller for monitoring hazardous gases within the exhaust system.

20. A system for monitoring gases at locations at atmospheric pressure around equipment where hazardous gases may be present, the system comprising:
at least one modular gas detection unit having:
at least one gas sensor cell having at least one gas sensor for detecting the composition and concentration of gases around the equipment;
a conduit operatively connected to each modular gas sensor cell for conveying gases from around the equipment to the gas sensor cell;
a gas flow system operatively connected to each gas sensor cell for moving gases through the conduit to each gas sensor cell;
a controller operatively connected to the modular gas detection unit for controlling the modular gas detection system and for receiving data signals from each gas sensor.

21. The system as in claim 20 wherein the at least one modular gas detection unit and controller each have wireless communication means for communicating data between the at least one modular gas detection unit and controller.

22. A pre-assembled plenum for configuration to equipment at atmospheric pressure to detect hazardous gases around the equipment, the pre-assembled plenum comprising at least two individual gas sensor cells operatively mounted within a plenum body, the gas sensor cells including:
at least one gas sensor for measuring hazardous gas within a sensor cell;
an inflow conduit connected to each sensor cell for drawing gases into a sensor cell, the inflow conduit extending from the plenum;
an outflow conduit connected to each sensor cell for expelling gases from a sensor cell, the outflow conduit extending from the plenum;
wherein each of the inflow and outflow conduits include means for connecting conduits to the inflow and outflow conduits.

23. The pre-assembled plenum as in claim 22 further comprising an individual gas flow system operatively connected to each sensor cell for drawing gas through each sensor cell.

24. A method of detecting hazardous gases around equipment at atmospheric pressure comprising the steps of:
a) conveying gases from around the equipment through a conduit to a gas detection system having at least one modular gas sensor cell having at least one gas sensor for detecting the composition of gases around the equipment wherein gases are conveyed through separate conduits to each modular gas sensor cell through separate conduits;
b) determining the composition of the gases from around the equipment and
c) initiating an alarm if an alarm condition is detected.

25. The method as in claim 24 further comprising the step of displaying or producing the alarm signal at the location where the hazardous gas is conveyed from.

26. The method as in claim 24 wherein gases are conveyed from at least two locations around the equipment and the step of displaying or producing the alarm signal at the location where the hazardous gas is conveyed from includes displaying a corresponding location of the origin of the alarm signal.

27. The system as in claim 3 wherein each gas sensor cell includes an independent gas flow system for moving gases through the conduits to each individual gas sensor, and the system further comprises:
an alarm system operatively connected to the controller, wherein the controller includes means for providing an audio and/or visual alarm if the controller detects an alarm condition, and wherein the alarm system provides distinct visual alarms that correspond to each gas sensor cell.
